(19) United States
(12) Patent Application Publication
(10) Pub. No.: US 2005/0272095 A1
(43) Pub. Date: Dec. 8, 2005

(54) Methods of identifying biomarkers

(75) Inventor: Weixun Wang, Mountain View, CA (US)

Correspondence Address: SHERIDAN ROSS PC, 1560 BROADWAY, SUITE 1200, DENVER, CO, 80202, US

(73) Assignee: PPD Biomarker Discovery Sciences, LLC, Wilmington, NC (US)

(21) Appl. No.: 11/134,022

(22) Filed: May 19, 2005

Related U.S. Application Data
(60) Provisional application No. 60/573,027, filed on May 19, 2004.

(51) Int. Cl. G01N 33/53; G01N 33/00

(52) U.S. Cl. 435/7.1; 436/86

(57) ABSTRACT

The invention provides methods of isolating and identifying biological markers of diabetes and metabolic syndrome and complications of these disease states. The biomarkers are useful in the evaluation, diagnosis and monitoring of these diseases.

### CROSS REFERENCE TO RELATED APPLICATION

This application claims priority under 35 U.S.C. § 119(e) to U.S. Provisional Patent Application Ser. No. 60/573,027, filed May 19, 2004, which is incorporated herein in its entirety by this reference.

### FIELD OF THE INVENTION

The invention relates to the glycation of molecules in a mammal and more particularly, to methods of isolating and identifying glycation sites on human proteins or peptides useful in diagnosing and monitoring the progression of elements of metabolic syndrome and/or diabetes.

### BACKGROUND OF THE INVENTION

The clustering of several metabolic and cardiovascular disease risk factors has been termed “metabolic syndrome.” The typical patient is characterized by abdominal obesity, insulin resistance or glucose intolerance, hyperlipidemia, hypertriglyceridemia, hypercholesterolemia, prothrombotic state (e.g., high fibrinogen or plasminogen activator inhibitor in the blood), proinflammatory state (e.g., elevated high-sensitivity C-reactive protein in the blood), dyslipidemia and hypertension. Metabolic syndrome has become increasingly common in the United States. It's estimated that about 20-25 percent of US adults suffer from one form or another of metabolic syndrome.

Metabolic syndrome is closely associated with a generalized metabolic disorder called insulin resistance, in which the body cannot use insulin efficiently. Metabolic syndrome may also be referred to as insulin resistance syndrome. Metabolic syndrome can precede the onset of type 2 diabetes or be associated with the disease. Diabetes is a metabolic disorder characterized by hyperglycemia, insulin resistance, and is often associated with other disorders such as obesity, hypertension, hyperlipidemia, hypertriglyceridemia and hypercholesterolemia as well as complications such as cardiovascular disease, retinopathy, neuropathy, and nephropathy. Nonenzymatic glycation of proteins with glucose occurs by a well known chemical route depicted in FIG. 1 resulting in advanced glycation endproducts or AGEs. AGEs are used as clinical biomarkers for diabetes, diabetic disease progression and complications that arise from diabetes. Although glycated hemoglobin is routinely used as a biomarker in clinical tests to monitor blood sugar levels over days or months, biomarkers that correlate with various complications of diabetes and metabolic syndrome and are still required for better diagnosis and treatments. Thus, chemical strategies for screening AGEs from diabetic mammals, or mammals exhibiting metabolic syndrome, are desired with the goal of discovering new biomarkers of metabolic syndrome, diabetes and/or complications of each.

### SUMMARY OF THE INVENTION

One embodiment of the present invention relates to a method of enriching advanced glycation endproducts (AGEs) in a biological sample comprising: (a) contacting a biological sample of a mammal with a compound comprising boron wherein AGEs in the biological sample form a complex with the boron; and, (b) separating the complex from the biological sample to produce an isolate having a higher concentration of AGEs than was present in the biological sample. The biological sample can include, but is not limited to, cerebrospinal fluid, serum, plasma, blood, urine, feces, sweat, mucus, prostatic fluid, saliva liver tissue, pancreatic tissue, spleen tissue and combinations thereof. In one aspect, the mammal is a human.

In one aspect of this embodiment, the compound is m-phenylboronic acid. In one aspect, the complex comprises a covalent bond between the boron and at least one oxygen present in the AGE.

In one aspect, the compound comprising boron is attached to a solid support. In this aspect of the invention, the separating can include washing the solid support to remove molecules that have not formed a complex with the compound comprising boron.

In one aspect of this embodiment, the concentration of AGEs in the isolate is at least 50-fold greater than the concentration of AGEs in the biological sample. In another aspect, the AGE comprises a mammalian protein.

In one aspect of this embodiment, the biological sample has been subjected to a treatment selected from the group consisting of reduction, alkylation, digestion, deglycosylation, desalting, lyophilization and combinations thereof. In another aspect, the contacting step and the separating step are conducted twice with respect to a biological sample.

In another aspect of this embodiment, the method further includes a step of washing the complex with a wash buffer comprising at least one chemical selected from an organic solvent, an aqueous solvent and an acidic solution.

In another aspect, the method further includes a step of eluting AGEs from the complex with the compound comprising boron. In this aspect, the step of eluting can include contacting the complex with a solution having a pH of less than about pH 6 to produce an eluate comprising at least one AGE. This method can further include analyzing a AGE in the eluate. Furthermore, in one aspect, the step of analyzing comprises subjecting at least a portion of the eluate to an analysis methodology selected from: protein sequencing, immunoassay, hybridization, enzyme assay, liquid chromatography (LC), mass spectroscopy (MS), gas chromatography (GC), electrospray ionization—time of flight (ESI-TOF) spectroscopy, matrix assisted laser desorption/ionization—time of flight (MALDI-TOF) spectroscopy, chromatographic separation, 2-D gel separation, immunoassay, competitive inhibition assays and combinations thereof, to produce a data set that can be compared to a reference data set. The reference data set may be produced by analyzing a biological sample from at least one mammal that has not been diagnosed with diabetes.

Another embodiment of the present invention relates to a method of producing an antibody useful in the clinical evaluation or progression of diabetes or metabolic syndrome comprising: (a) contacting a biological sample of a mammal known to have diabetes or metabolic syndrome with a compound comprising boron wherein AGEs in the biological sample form a complex with the boron; (b) separating the complex from the biological sample to produce an isolate having a higher concentration of AGEs than was present in the biological sample; (c) immunizing a host animal with AGEs; and, (d) isolating antibodies to the AGEs from the host animal.

Yet another embodiment of the present invention relates to a method of diagnosing diabetes or metabolic syndrome in a mammal comprising analyzing a biological sample from the mammal for a biomarker of diabetes or metabolic syndrome identified by the method comprising: (a) contacting a biological sample of a mammal known to have diabetes or metabolic syndrome with a compound comprising boron wherein AGEs in the biological sample form a complex with the boron; (b) separating the complex from the biological sample to produce an isolate having a higher concentration of AGEs than was present in the biological sample; and, (c) analyzing the AGEs for the presence of biological markers that correlate with the incidence of diabetes or metabolic syndrome.

Another embodiment of the present invention relates to a method of identifying a therapeutic target for the treatment or prevention of diabetes comprising: (a) contacting a biological sample of a mammal known to have diabetes or metabolic syndrome with a compound comprising boron wherein AGEs in the biological sample form a complex with the boron; (b) separating the complex from the biological sample to produce an isolate having a higher concentration of AGEs than was present in the biological sample; and, (c) analyzing the AGEs for the presence of biological markers that correlate with the incidence of diabetes or metabolic syndrome.

Another embodiment of the present invention relates to a method of monitoring the progression of diabetes or metabolic syndrome in a mammal comprising analyzing a biological sample from the mammal for a biomarker of diabetes or metabolic syndrome identified by the method comprising: (a) contacting a biological sample of a mammal known to have diabetes or metabolic syndrome with a compound comprising boron wherein AGEs in the biological sample form a complex with the boron; (b) separating the complex from the biological sample to produce an isolate having a higher concentration of AGEs than was present in the biological sample; and, (c) analyzing the AGEs for the presence of biological markers that correlate with the incidence of diabetes or metabolic syndrome.

### BRIEF DESCRIPTION OF THE DRAWINGS

FIG. 1 shows the chemical scheme of glycation of a protein (R) by formation of the Amadori product followed by dehydration and rearrangement to produce the final advanced glycation endproduct (AGE).

FIG. 2 depicts the formation of a covalent bond between m-aminophenylboronic acid and a glycated protein molecule.

FIG. 3 shows a scheme for one preferred method of the present invention of enriching AGEs.

FIG. 4 shows MALDI spectra of glycated human serum albumin and non-glycated control isolated by the enrichment processes of the present invention.

FIG. 5 demonstrates boronic acid bead enrichment of glycated albumin peptides. A) LC-MS profile of tryptic-digested human albumin peptides with 1% of digested glycated albumin peptides spiked in; and, B) LC-MS profile of enriched glycated albumin peptides.

FIG. 6 is the mass chromatograms showing the relative enrichment of glycated albumin peptides. The upper panel shows that three glycated albumin peptides were greatly enriched, while the lower panel shows that three non-glycated albumin peptides were almost completely removed by the selective enrichment process.

FIG. 7 shows the enrichment of glycated peptides in diabetic serum sample on boronic acid beads. A) LC-MS profile of tryptic-digested diabetic serum proteome. B) LC-MS profile of enriched glycated peptides.

FIG. 8 shows a MS/MS spectrum of a peptide with glycation on a lysine residue in apolipoprotein AII, having the sequence VKSPELQAEAK.

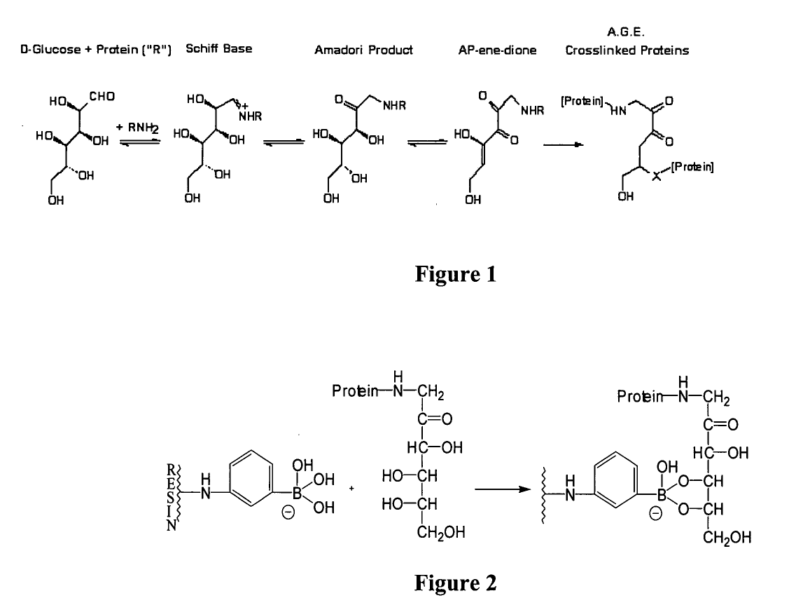

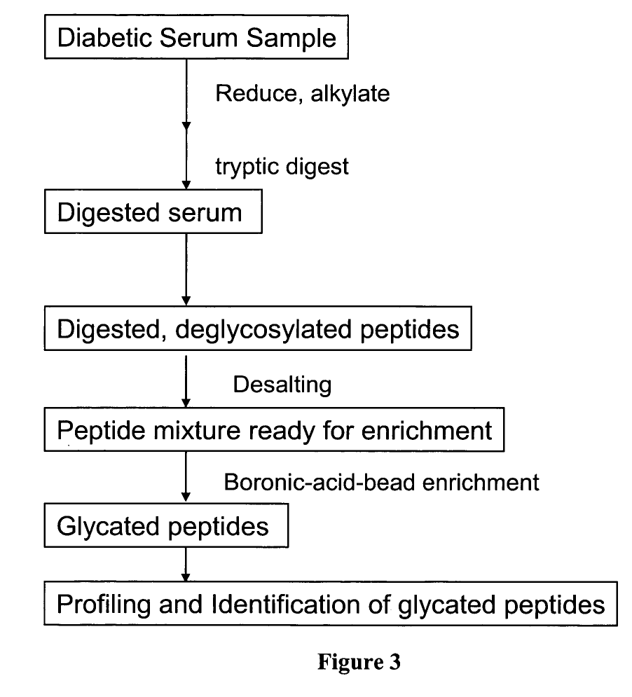

### DETAILED DESCRIPTION OF THE INVENTION

The present invention provides processes for enriching and identifying biomarkers of metabolic syndrome, diabetes, and/or complications of each.

The biomarkers of the present invention are AGEs, or the polypeptide comprising an AGE, found in an animal. These AGEs are first enriched to easily detectable levels within biological samples taken from a mammal. Control mammals are initially tested and thereafter, mammals suspected of having diabetes or metabolic syndrome, or suspected of having a propensity of developing diabetes or metabolic syndrome, are tested using the methods of the present invention. Preferably, the mammal is a human. The biological sample includes a sample from any body fluid such as cerebrospinal fluid, serum, plasma, blood, urine, feces, sweat, mucus, prostatic fluid, saliva or fluid from tissues such as liver, pancreas or spleen. Preferably, the biological sample is serum.

The biological sample is contacted with a boron containing compound that forms a complex with the glycan moiety in an AGE in a biological sample. Preferably, the boron containing compound is m-phenylboronic acid. The boron containing compound may be attached to a solid support to aid in separation of the complex from other biological molecules within the biological sample. Preferably, the boron containing compound is attached to a resin such as polyacrylamide beads which are available commercially.

The complex formed between the AGEs and the boron may include any form of molecular association such as ionic bonds or covalent bonds. Preferably, the boron containing compound forms a covalent bond with the AGEs. FIG. 2 shows a covalent complex formed between a protein and m-phenylboronic acid bound to a sold support.

The contacting is conducted under conditions conducive to the formation of the complex between the boron containing compound and the glycan residue of the AGE. These conditions will necessarily be slightly different depending upon the boron containing compound and the AGE present in the biological sample. Typically the biological sample is incubated in the presence of the boron containing compound at a temperature between about 20° C. and 80° C. and is preferably conducted at room temperature without specifically heating or cooling the biological sample. The incubation is typically conducted for a time between about one minute and about four hours. Preferably, the incubation of the biological sample with the boron containing compound is conducted for about 30 minutes or less.

Optionally, the biological sample can be subjected to different pre-treatments prior to contact with the boron containing compound. For example, the biological sample may be reduced, alkylated, completely- or partially-digested, deglycosylated, desalted, lyophilized or any combination of these pre-treatments. Reduction can be accomplished by any suitable reducing agent. Similarly, alkylation can be accomplished by any suitable alkylating agent. Digestion is preferably accomplished by contacting the biological sample with a solution containing trypsin. The removal of N-linked and O-linked glycans from the biological sample may be accomplished by mixing the biological sample with a mixture of glycosidases at about 37° C. for a period of time less than about 48 hours. Preferably, the biological sample is mixed with a mixture of five N-glycosidase and O-glycosidase enzymes that is commercially available (PROZYME™). Preferably, the biological sample is subjected to reduction, akylation, tryptic digestion, deglycosylation, desalting and lyophylization prior to contact with the boron containing compound.

The complex between the AGEs and the boron containing compounds is then separated from the remainder of the biological sample to isolate and enrich the AGEs. This separation results in the production of at least one AGE complexed with a boron containing compound. The separation can be effected by any process that physically separates the complex from the remaining components of the biological sample. For example, in the instance that the boron containing compound is attached to a solid support, the complex can be centrifuged, filtered, or separated chromatographically from the remainder of the sample. Preferably the separation technique is applied to the biological sample two or more successive times to remove the vast majority of the complex from the remainder of the biological sample. For example, the complex is preferably washed at least twice with a wash buffer containing a mixture of water and an organic solvent such as acetonitrile at a pH above about pH 7.

Following separation from the remainder of the biological sample, the complex may be contacted with any chemical or condition that causes the complex to dissolve, thereby releasing AGEs present from the complex with the boron containing compound. The AGEs may then be collected for further study including determining the identity of the polypeptide comprising the AGE, the point of glycation and the role of these glycated proteins or peptides in diabetes, metabolic syndrome and/or complications of each.

The complex may be dissolved by contacting the complex with an elution buffer having a pH below about pH 6. The elution buffer may be either organic or aqueous and is preferably a mixture of water and an organic solvent and is most preferably a mixture of water and methanol. The elution buffer preferably contains a pH adjusting compound or pH buffering composition and preferably contains formic acid in a concentration of below about 1M. The elution buffer may be applied to the complex in any suitable manner to release AGEs present from the boron containing compound. Preferably, the complex is contacted with elution buffer and agitated to dissolve the complex and release the AGEs. In a preferred embodiment, the boron containing compound is bound to a solid support and an elution buffer containing water, methanol and formic acid is applied to the complex, vortexed and centrifuged to produce a supernatant isolate containing the AGEs.

The conditions used to dissolve the complex are chosen to maximize the release of the AGEs from the complex. However, the conditions should not be so harsh as to modify or destroy the AGEs or to impede any intended additional study of the AGE. Typically, the elution buffer is applied to the complex at a temperature between about room temperature and about 50° C., and most preferably, the elution buffer is applied to the complex at about room temperature. The elution buffer may be incubated with the dissolving complex for a period of time between about five seconds and about 6 hours. Preferably, the elution buffer is passed over the complex, making contact for about 30 seconds. This elution step may be repeated two or more successive times to secure the release of as many AGEs as possible from the complex.

The AGEs collected in the elution buffer in a concentration of AGE compounds that is substantially higher than the concentration of the AGEs present in the original biological sample. The enrichment of AGE molecules over the concentration found in the biological sample may be a greater than about 100-fold increase. As used herein, the term “fold increase” refers to the relative increase or decrease in the level of an AGE in one sample or a set of samples compared to another sample or set of samples. A positive fold change indicates an increase in the level of an AGE while a negative fold change indicates a decrease in the level of an AGE. Typically, the enrichment is between about 50-fold and about 300-fold. More typically, the enrichment is between about 100-fold and about 200-fold.

Further study of the isolated AGEs to determine the identity of the polypeptide comprising the AGE may include analysis such as protein sequencing, immunoassays, hybridization and enzyme assays, liquid chromatography (LC), mass spectroscopy (MS), gas chromatography (GC), electrospray ionization—time of flight (ESI-TOF) spectroscopy, matrix assisted laser desorption/ionization—time of flight (MALDI-TOF) spectroscopy, chromatographic separations, 2-D gel separations, binding assays such as immunoassays, competitive inhibition assays, and so on. Any effective method in the art for measuring the presence/absence, level or activity of a metabolite, polypeptide or polynucleotide is included in the invention. It is within the ability of one of ordinary skill in the art to determine which method would be most appropriate for analyzing an AGE.

The level of a biomarker of the present invention can be measured by mass spectrometry, which allows direct measurements of analytes with high sensitivity and reproducibility. A number of mass spectrometric methods are available. Electrospray ionization (ESI), for example, allows quantification of differences in relative concentration of various species in one sample against another; absolute quantification is possible by normalization techniques such as using an internal standard. MALDI or the related SELDI™ technology (Ciphergen, Inc.) also could be used to make a determination of whether a biomarker was present, and the relative or absolute level of the biomarker. Mass spectrometers that allow time-of-flight (TOF) measurements have high accuracy and resolution and are able to measure low abundant species, even in complex matrices.

Quantification can be based on derivatization in combination with isotopic labeling, referred to as isotope coded affinity tags (“ICAT”). In this and other related methods, a specific amino acid in two samples is differentially and isotopically labeled and subsequently separated from peptide background by solid phase capture, wash and release. The intensities of the molecules from the two sources with different isotopic labels can then be accurately quantified with respect to one another.

In addition, one- and two-dimensional gels have been used to separate proteins and quantify gels spots by silver staining, fluorescence or radioactive labeling. These differently stained spots have been detected using mass spectrometry, and identified by tandem mass spectrometry techniques.

As will be appreciated by one of skill in the art, many other technologies for separation and analysis may be used in connection with mass spectrometry. For example, a wide selection of separation columns is commercially available. In addition, separations may be performed using custom chromatographic surfaces such as a bead on which a biomarker-specific reagent has been immobilized. A biomarker retained on the media subsequently may be eluted for analysis by mass spectrometry.

Analysis by liquid chromatography-mass spectrometry produces a mass intensity spectrum, the peaks of which represent various components of the sample, each component having a characteristic mass-to-charge ratio (m/z) and retention time (RT). The presence of a peak with the m/z and RT of a biomarker indicates that the biomarker is present. The peak representing a biomarker may be compared to a corresponding peak from another spectrum, such as from a control sample, to obtain a relative measurement. Any normalization technique in the art, such as an internal standard, may be used when a quantitative measurement is desired. “Deconvoluting” software is available to separate overlapping peaks. The retention time depends to some degree on the conditions employed in performing the liquid chromatography separation. The mass spectrometer preferably provides high mass accuracy and high mass resolution. The mass accuracy of a well-calibrated Micromass TOF instrument, for example, is reported to be approximately 2 mDa, with resolution m/Δm exceeding 5000.

The level of biomarkers may be determined using a standard immunoassay, such as sandwiched ELISA using matched antibody pairs and chemiluminescent detection. Any antibody suitable for binding to a biomarker of the invention can be used. Standard protocols and data analysis are used to determine the biomarker concentrations from the assay data.

Antibodies that bind to a biomarker of the invention can be used to assay a tissue sample, such as liver, pancreatic, cardiac or optic tissue, for biomarkers. The antibodies can specifically bind to the biomarker, if any, present in the tissue sections and allow the localization of the biomarker in the tissue. Similarly, antibodies labeled with a radioisotope may be used for in vivo imaging or treatment applications.

A number of the assays discussed above employ a reagent that specifically binds to the biomarker. Any molecule that is capable of specifically binding to a biomarker is included within the invention. In some embodiments, the binding molecules are antibodies or antibody fragments. In other embodiments, the binding molecules are non-antibody species. Thus, for example, the binding molecule may be an enzyme for which the biomarker is a substrate. The binding molecules may recognize any epitope of the targeted biomarkers.

The chromatographic separation techniques described above also may be coupled to an analytical technique other than mass spectrometry such as fluorescence detection of tagged molecules, NMR, capillary UV, evaporative light scattering or electrochemical detection.

The biomarkers of the invention are useful for diagnosing diabetes and metabolic syndrome, determining the extent and/or severity of the disease, monitoring progression of the disease, selecting a therapeutic treatment and/or determining the response to therapy. The methods include determining the level of a biomarker in a biological sample.

These methods comprise obtaining a biological sample from a subject suspected of having diabetes or metabolic syndrome, or at risk for developing these disease states, detecting the level or activity of a biomarker of the invention in the sample, and comparing the result to the level or activity of the biomarker in a non-subject sample, or to a reference range or value. Either an increased or decreased level or activity of a biomarker as compared to a baseline or normal level is indicative that the subject: (a) has or is at risk for developing diabetes or metabolic syndrome, (b) progression to diabetes or metabolic syndrome, (c) should use a particular therapeutic treatment, or (d) is likely to respond to a particular treatment, depending upon the particular biomarker being measured. Fragments, precursors, successors and modified versions of such biomarkers, polypeptides having substantial homology to such biomarkers are suitable biomarkers for diagnostic methods of the invention.

The term “biomarker” or “marker”, as used herein, can refer to polypeptide or metabolite thereof. In addition, the term “biomarker” can be generally used to refer to any portion of such a polypeptide that can identify or correlate with the full-length polypeptide, for example, in an assay of the invention. Biomarkers also include any precursors and successors of polypeptides of the invention, as well as polypeptides substantially homologous to polypeptides of the invention. Accordingly, a biomarker useful in the present invention is any polypeptide or metabolite, the expression of which is regulated (up or down) in a patient with a condition (e.g., metabolic syndrome) as compared to a normal control. Selected sets of one, two, three, and more preferably several more of the biomarkers of this invention (up to the number equivalent to all of the biomarkers, including any intervening number, in whole number increments, e.g., 1, 2, 3, 4, 5, 6 . . . ) can be used as end-points for rapid diagnostics or prognostics for metabolic syndrome and/or diabetes, and/or as targets for the development of therapeutic drugs and strategies for the treatment of these disease states. In one embodiment, larger numbers of the biomarkers identified herein are used in a diagnostic assay of the invention, since the accuracy of the assay improves as the number of biomarkers screened increases.

A normal level of a biomarker can be determined in a variety of ways. For example, if a patient history is known, a baseline level of the biomarker can be determined and higher or lower levels can be determined. Alternatively, a normal level can be based on the level for a healthy individual in a given population. That is, a normal level can be based on a population having similar characteristics (e.g., age, sex, race, medical history) as the patient in question.

More specifically, according to the present invention, a “baseline level” is a control level, and in some embodiments, a normal level, of biomarker expression or activity against which a test level of biomarker expression or biological activity (i.e., in the test sample) can be compared. Therefore, it can be determined, based on the control or baseline level of biomarker expression or biological activity, whether a sample to be evaluated has a measurable increase, decrease, or substantially no change in biomarker expression or biological activity, as compared to the baseline level. In one aspect, the baseline level can be indicative of a sample expected from a normal (i.e., healthy, negative control) individual. Therefore, the term “negative control” used in reference to a baseline level of biomarker expression or biological activity typically refers to a baseline level established in a sample from the patient or from a population of individuals which is believed to be normal. In another embodiment, a baseline can be indicative of a positive diagnosis of diabetes or metabolic syndrome. Such a baseline level, also referred to herein as a “positive control” baseline, refers to a level of biomarker expression or biological activity established in a sample from the patient, another patient, or a population of individuals, wherein the sample was believed to be indicative of diabetes or metabolic syndrome. In yet another embodiment, the baseline level can be established from a previous sample from the patient being tested, so that the condition of a patient can be monitored over time and/or so that the efficacy of a given therapeutic protocol can be evaluated over time. Methods for detecting biomarker expression or biological activity are described in detail above.

The method for establishing a baseline level of biomarker expression or activity is selected based on the sample type, the status of the patient to be evaluated, and, as discussed above, the focus or goal of the assay (e.g., diagnosis, staging, monitoring). Preferably, the method is the same method that will be used to evaluate the sample in the patient. In a most preferred embodiment, the baseline level is established using the same sample type as the sample to be evaluated.

In one embodiment, the baseline level of biomarker expression or biological activity is established in an autologous control sample obtained from the patient. According to the present invention, and as used in the art, the term “autologous” means that the sample is obtained from the same patient from which the sample to be evaluated is obtained. The control sample should preferably be the same sample type as the sample to be evaluated, such that the control sample serves as the best possible baseline for the sample to be evaluated.

One method for establishing a baseline level of biomarker expression or biological activity is to establish a baseline level of biomarker expression or biological activity from at least one measurement of biomarker expression or biological activity in a previous sample from the same patient. Such a sample is an autologous sample, but is taken from the patient at a different time point than the sample to be tested. Preferably, the previous sample(s) were of the same sample type as the sample to be presently evaluated. In one embodiment, the previous sample resulted in a negative diagnosis. In this embodiment, a new sample is evaluated periodically (e.g., at annual physicals), and as long as the patient is determined to be negative, an average or other suitable statistically appropriate baseline of the previous samples can be used as a “negative control” for subsequent evaluations. For the first evaluation, an alternate control can be used, as described below, or additional testing may be performed to confirm an initial negative diagnosis, if desired, and the value for biomarker expression or biological activity can be used thereafter. This type of baseline control is frequently used in other clinical diagnosis procedures where a “normal” level may differ from patient to patient and/or where obtaining an autologous control sample at the time of diagnosis is not possible, not practical or not beneficial.

In another embodiment, the previous sample from the patient resulted in a positive diagnosis. In this embodiment, the baseline provided by the previous sample is effectively a positive control, and the subsequent samplings of the patient are compared to this baseline to monitor the progress of the patient and/or to evaluate the efficacy of a treatment which is being prescribed. In this embodiment, it may also be beneficial to have a negative baseline level of biomarker expression or biological activity (i.e., a normal baseline control), so that a baseline for remission or regression can be set.

It will be clear to those of skill in the art that some samples to be evaluated will not readily provide an obvious autologous control sample, or it may be determined that collection of autologous control samples is too invasive and/or causes undue discomfort to the patient. In these instances, an alternate method of establishing a baseline level of biomarker expression or biological activity can be used, examples of which are described below.

Another method for establishing a baseline level of biomarker expression or biological activity is to establish a baseline level of biomarker expression or biological activity from control samples, and preferably control samples that were obtained from a population of matched individuals. It is preferred that the control samples are of the same sample type as the sample type to be evaluated for biomarker expression or biological activity. According to the present invention, the phrase “matched individuals” refers to a matching of the control individuals on the basis of one or more characteristics which are suitable for the type of cell or tumor growth to be evaluated. For example, control individuals can be matched with the patient to be evaluated on the basis of gender, age, race, or any relevant biological or sociological factor that may affect the baseline of the control individuals and the patient (e.g., preexisting conditions, consumption of particular substances, levels of other biological or physiological factors). To establish a control or baseline level of biomarker expression or biological activity, samples from a number of matched individuals are obtained and evaluated for biomarker expression or biological activity. The sample type is preferably of the same sample type to be evaluated in the test patient. The number of matched individuals from whom control samples must be obtained to establish a suitable control level (e.g., a population) can be determined by those of skill in the art, but should be statistically appropriate to establish a suitable baseline for comparison with the patient to be evaluated (i.e., the test patient). The values obtained from the control samples are statistically processed using any suitable method of statistical analysis to establish a suitable baseline level using methods standard in the art for establishing such values.

A baseline such as that described above can be a negative control baseline, such as a baseline established from a population of apparently normal control individuals. Alternatively, as discussed above, such a baseline can be established from a population of individuals that have been positively diagnosed with diabetes or metabolic syndrome, so that one or more baseline levels can be established for use in diagnosing the patient to be evaluated.

It will be appreciated by those of skill in the art that a baseline need not be established for each assay as the assay is performed but rather, a baseline can be established by referring to a form of stored information regarding a previously determined baseline level of biomarker expression for a given control sample, such as a baseline level established by any of the above-described methods. Such a form of stored information can include, for example, but is not limited to, a reference chart, listing or electronic file of population or individual data regarding “normal” (negative control) or positive biomarker expression; a medical chart for the patient recording data from previous evaluations; or any other source of data regarding baseline biomarker expression that is useful for the patient to be diagnosed.

After the level of biomarker expression or biological activity is detected in the sample to be evaluated, such level is compared to the established baseline level of biomarker expression or biological activity, determined as described above. Also, as mentioned above, preferably, the method of detecting used for the sample to be evaluated is the same or qualitatively and/or quantitatively equivalent to the method of detecting used to establish the baseline level, such that the levels of the test sample and the baseline can be directly compared. In comparing the test sample to the baseline control, it is determined whether the test sample has a measurable decrease or increase in biomarker expression or biological activity over the baseline level, or whether there is no statistically significant difference between the test and baseline levels. After comparing the levels of biomarker expression or biological activity in the samples, the final step of making a diagnosis, or monitoring, can be performed as discussed above.

This method of diagnosis can be used specifically to determine the prognosis of the patient or to determine the susceptibility of the patient to a therapeutic treatment. In some embodiments, the method may be useful to monitor the progress of a patient undergoing therapeutic treatment for a tumor.

Each biomarker may be considered individually, although it is within the scope of the invention to provide combinations of two or more biomarkers for use in the methods and compositions of the invention. The use of such combinations typically will increase the confidence of the analysis. For example, a panel of biomarkers may include biomarkers that are increased in level or activity in diabetes or metabolic syndrome subject samples as compared to non-subject samples, biomarkers that are decreased in level or activity in diabetes or metabolic syndrome subject samples as compared to non-subject samples, or a combination thereof. A panel of biomakers may include one or more biomarkers of the invention. The panel of biomarkers may also be evaluated with other clinical indicia of diabetes or metabolic syndrome. The biomarker may be detected in any biological sample obtained from the subject, by any suitable method known in the art or as described herein.

Preferred biomarkers of the present invention are provided in Table 1. Reference to a protein's database Accession number(s), or other information regarding the biomarkers in Table 1, is hereby an express incorporation by reference of the entire sequence of the protein. Table 1 identifies 38 biomarkers of available name that were identified using the enrichment techniques of the present invention. Each biomarker polypeptide is identified in Table 1 by the identification number from the National Center for Biotechnology Information (NCBI) sequence database (Accession # and gi #) and by the name, sequence and/or partial sequence of the peptide biomarker as contained in the NCBI queried database.

TABLE 1
Preferred biomarkers of the present invention.

| | | | |
|---|---|---|---|
| Accession | | | |
| Number | Protein description | Gi | Peptide Sequence |

| | | | |
|---|---|---|---|
| NP_000030.1 | apolipoprotein A-I precursor [Homo sapiens] | 4557321 | LSPLGEEMR |
| NP_000030.1 | apolipoprotein A-I precursor [Homo sapiens] | 4557321 | QKLHELQEK |
| NP_000030.1 | apolipoprotein A-I precursor [Homo sapiens] | 4557321 | LEALKENGGAR |
| NP_000030.1 | apolipoprotein A-I precursor [Homo sapiens] | 4557321 | ATEHLSTLSEK |
| NP_000030.1 | apolipoprotein A-I precursor [Homo sapiens] | 4557321 | VQPYLDDFQK |
| NP_000030.1 | apolipoprotein A-I precursor [Homo sapiens] | 4557321 | THLAPYSDELR |
| NP_000030.1 | apolipoprotein A-I precursor [Homo sapiens] | 4557321 | VQPYLDDFQKK |
| NP_000030.1 | apolipoprotein A-I precursor [Homo sapiens] | 4557321 | DYVSQFEGSALGK |
| NP_000030.1 | apolipoprotein A-I precursor [Homo sapiens] | 4557321 | KWQEEMELYR |
| NP_000030.1 | apolipoprotein A-I precursor [Homo sapiens] | 4557321 | KWQEEMELYR |
| NP_000030.1 | apolipoprotein A-I precursor [Homo sapiens] | 4557321 | AKVQPYLDDFQK |
| NP_000030.1 | apolipoprotein A-I precursor [Homo sapiens] | 4557321 | DSGRDYVSQFEGSALGK |
| NP_005134.1 | haptoglobin [Homo sapiens] | 4826762 | QLVEIEK |
| NP_005134.1 | haptoglobin [Homo sapiens] | 4826762 | GSFPWQAK |
| NP_005134.1 | haptoglobin [Homo sapiens] | 4826762 | VGYVSGWGR |
| NP_005134.1 | haptoglobin [Homo sapiens] | 4826762 | KQLVEIEK |
| NP_005134.1 | haptoglobin [Homo sapiens] | 4826762 | TEGDGVYTLNDKK |
| NP_001634.1 | apolipoprotein A-II precursor [Homo sapiens] | 4502149 | SKEQLTPLIK |
| NP_001634.1 | apolipoprotein A-II precursor [Homo sapiens] | 4502149 | VKSPELQAEAK |
| NP_001634.1 | apolipoprotein A-II precursor [Homo sapiens] | 4502149 | SKEQLTPLIK |
| NP_001634.1 | apolipoprotein A-II precursor [Homo sapiens] | 4502149 | VKSPELQAEAK |
| NP_001054.1 | transferrin [Homo sapiens] | 4557871 | KASYLDCIR |
| NP_001054.1 | transferrin [Homo sapiens] | 4557871 | KDSGFQMNQLR |
| NP_000598.1 | orosomucoid 1 precursor; Orosomucoid-1 (alpha-1-acid | 9257232 | SDVVYTDWK |
| | glycoprotein-1); | | |
| NP_000604.1 | hemopexin [Homo sapiens] | 11321561 | GDKVWVYPPEK |
| NP_000604.1 | hemopexin [Homo sapiens] | 11321561 | GDKVWVYPPEKK |
| NP_000604.1 | hemopexin [Homo sapiens] | 11321561 | GDKVWVYPPEKK |
| P01011 | Alpha-1-antichymotrypsin precursor (ACT) | 112874 | GKITDLIK |
| NP_000005.1 | alpha 2 macroglobulin precursor [Homo sapiens] | 4557225 | SIYKPGQTVK |
| NP_000005.1 | alpha 2 macroglobulin precursor [Homo sapiens] | 4557225 | LVDGKGVPIPNK |
| P01860 | Ig gamma-3 chain C region (Heavy chain disease | 121045 | VSNKALPAPIEK |
| | protein) (HDC) | | |
| | similar to Elongation factor 1-delta (EF-1-delta) | | |
| XP_069791.1 | (Antigen NY-CO-4) [Homo sapiens] | 17448940 | RVVQELQQAISK |
| P01876 | Ig alpha-1 chain C region | 113584 | GFSPKDVLVR |
| Q13972 | Guanine nucleotide releasing protein (GNRP) (Ras- | 13124259 | VTVPQMIK |
| | specific nucleotide exchange factor CDC25) | | |
| XP_097736.1 | chromosome 20 open reading frame 82 [Homo sapiens] | 18592401 | TCPAAPAPTPLR |
| XP_212172.1 | hypothetical protein [Homo sapiens] | 27478522 | TPGRNLR |
| NP_078789.1 | FYVE and coiled-coil domain containing 1 [Homo | 13470092 | EAMKAQMAEK |
| | sapiens] | | |
| NP_000280.1 | Phosphofructokinase, muscle [Homo sapiens] | 4505749 | DVTKAMDEK |

As used herein, the term “polypeptide” refers to a polymer of amino acid residues, and includes a sufficient number of amino acids to identify the polypeptide as a biomarker in the present invention. Therefore, a polypeptide can include a peptide, an oligopeptide, a protein, and may be composed of two or more polypeptide chains. A polypeptide can be linear or branched. A polypeptide can comprise modified amino acid residues, amino acid analogs or non-naturally occurring amino acid residues and can be interrupted by non-amino acid residues. Included within the definition are amino acid polymers that have been modified, whether naturally or by intervention, such as formation of a disulfide bond, glycosylation, lipidation, methylation, acetylation, phosphorylation, or by manipulation, such as conjugation with a labeling component.

As used herein, the term “homologue” is used to refer to a polypeptide which differs from a naturally occurring polypeptide by one or more minor modifications or mutations to the naturally occurring polypeptide, but which maintains the overall basic protein and side chain structure of the naturally occurring form (i.e., such that the homologue is identifiable as being related to the wild-type polypeptide). Such changes include, but are not limited to: changes in one or a few amino acid side chains; changes one or a few amino acids, including deletions (e.g., a truncated version of the protein or peptide) insertions and/or substitutions; changes in stereochemistry of one or a few atoms; and/or minor derivatizations, including but not limited to: methylation, farnesylation, geranyl geranylation, glycosylation, carboxymethylation, phosphorylation, acetylation, myristoylation, prenylation, palmitation, and/or amidation. A homologue can have either enhanced, decreased, or substantially similar properties as compared to the naturally occurring polypeptide. Homologues can include synthetically produced homologues, naturally occurring allelic variants of a given protein or domain, or homologous sequences from organisms other than the organism from which the reference polypeptide was derived.

As used herein, two polypeptides are “substantially homologous” or “homologues” when there is at least 70% homology, at least 80% homology, at least 90% homology, at least 95% homology or at least 99% homology between their amino acid sequences, or when polynucleotides encoding the polypeptides are capable of forming a stable duplex with each other. As used herein, unless otherwise specified, reference to a percent (%) identity refers to an evaluation of homology which is performed using: (1) a BLAST 2.0 Basic BLAST homology search using blastp for amino acid searches, blastn for nucleic acid searches, and blastX for nucleic acid searches and searches of translated amino acids in all 6 open reading frames, all with standard default parameters, wherein the query sequence is filtered for low complexity regions by default (described in Altschul, S. F., Madden, T. L., Schaaffer, A. A., Zhang, J., Zhang, Z., Miller, W. & Lipman, D. J. (1997) “Gapped BLAST and PSI-BLAST: a new generation of protein database search programs.” Nucleic Acids Res. 25:3389, incorporated herein by reference in its entirety); (2) a BLAST 2 alignment (using the parameters described below); (3) and/or PSI-BLAST with the standard default parameters (Position-Specific Iterated BLAST). It is noted that due to some differences in the standard parameters between BLAST 2.0 Basic BLAST and BLAST 2, two specific sequences might be recognized as having significant homology using the BLAST 2 program, whereas a search performed in BLAST 2.0 Basic BLAST using one of the sequences as the query sequence may not identify the second sequence in the top matches. In addition, PSI-BLAST provides an automated, easy-to-use version of a “profile” search, which is a sensitive way to look for sequence homologues. The program first performs a gapped BLAST database search. The PSI-BLAST program uses the information from any significant alignments returned to construct a position-specific score matrix, which replaces the query sequence for the next round of database searching. Therefore, it is to be understood that percent identity can be determined by using any one of these programs.

As used herein, a “fragment” of a polypeptide refers to a single or a plurality of amino acid residues comprising an amino acid sequence that has at least 5 contiguous amino acid residues, at least 10 contiguous amino acid residues, at least 20 contiguous amino acid residues, at least 30 contiguous amino acid residues, at least 40 contiguous amino acid residues, at least 50 contiguous amino acid residues, at least 60 contiguous amino acid residues, at least 70 contiguous amino acid residues, at least 80 contiguous amino acid residues, at least 90 contiguous amino acid residues, or at least 100 contiguous amino acid residues of a sequence of the polypeptide, or any number of residues between 5 and 100, in whole number increments.

The present invention also includes antibodies and antibody fragments that specifically bind to a biomarker identified and isolated by the enrichment method described above. The invention also provides antibodies that specifically bind to a polypeptide having substantial homology with a polypeptide biomarker identified and isolated by the enrichment method described above. The invention also provides antibodies that specifically bind to a component that is a fragment, modification, precursor or successor of a biomarker identified and isolated by the enrichment method described above.

The antibodies of the invention may be prepared by any suitable means known in the art. For example, antibodies may be prepared by immunizing an animal host with an AGE or an immunogenic fragment thereof (conjugated to a carrier, if necessary). Adjuvants such as Freund's adjuvant optionally may be used to increase the immunological response. Sera containing polyclonal antibodies with high affinity for the antigenic determinant can then be isolated from the immunized animal and purified.

Alternatively, antibody-producing tissue from the immunized host can be harvested and a cellular homogenate prepared from the organ can be fused to cultured cancer cells. Hybrid cells which produce monoclonal antibodies specific for a biomarker can be selected. Alternatively, the antibodies of the invention can be produced by chemical synthesis or by recombinant expression. For example, a polynucleotide that encodes the antibody can be used to construct an expression vector for the production of the antibody. The antibodies of the present invention can also be generated using various phage display methods known in the art.

Methods for identifying and producing antibodies and antibody fragments specific for an analyte are well known. Examples of other methods used to identify the binding molecules include binding assays with random peptide libraries, such as phage display, and design methods based on an analysis of the structure of the biomarker.

Additional objects, advantages, and novel features of this invention will become apparent to those skilled in the art upon examination of the following examples thereof, which are not intended to be limiting.

### EXAMPLES

### Example 1

### Preparation of a Human Serum Sample

Human albumin was incubated overnight with different concentrations of glucose under physiological conditions (pH 7.5, 37° C.). Removal of six major serum proteins from the serum proteome sample by antibody column (Agilent Technologies) was performed on a PerSeptive Vision™ HPLC system coupled with a UV detector and a fraction collector. Thirty-five microliter of diabetic serum sample was carried through each removal cycle and the serum (or plasma) proteins that don't bind to antibody column were collected and further reduced, alkylated and tryptic digested.

The digested proteome mixture was desalted with a RapidTrace® robotic desalting system and lyophilized. It was then re-dissolved in 10 mM phosphate buffer at pH 7.0 and ready to be de-glycosylated. A mixture of five N-glycosidase and O-glycosidase (ProZyme™) was added, and the system was incubated at 37° C. for 16 hours. A second dose of the enzyme mixture was added at the end of the first 16 hours and the incubation was prolonged for another 16 hours. After enzyme de-glycosylation, the peptide mixture was desalted to remove free glycans that could interfere with later enrichment process (with Rapidtrace®) and lyophilized again.

### Example 2

### Enrichment of AGEs Over a Boron Containing Compound

Enrichment by m-phenylboronic acid beads (Pierce): 200 μL of polyacrylamide based boronic acid beads were washed in a Spin-X® (Corning) mini-filtration device with 500 μL of 0.2 M ammonium acetate in water at pH 8.8 twice by mixing and centrifugation. The dried peptide mixture was dissolved also with 500 μL of 0.2 M ammonium acetate in water at pH 8.8 and applied to the washed boronic acid beads in the Spin-X® device. The capture of glycated peptides with boronic acid beads was achieved by vortexing the mixture for 30 minutes at room temperature and the non-glycated peptides was removed as flow through by centrifugation. The beads were then washed twice: first with 500 μL of 40% acetonitrile, 0.2 M ammonium acetate in water at pH 8.8, then with 500 μL of 80% acetonitrile, 0.2 M ammonium acetate at pH 8.8. The captured glycated peptides were released by washing the beads with elution buffer: 60% methanol, 0.1 M formic acid in water. The elution buffer contained enriched glycated peptides.

### Example 3

### Analysis of Recovered AGEs by MALDI-TOF

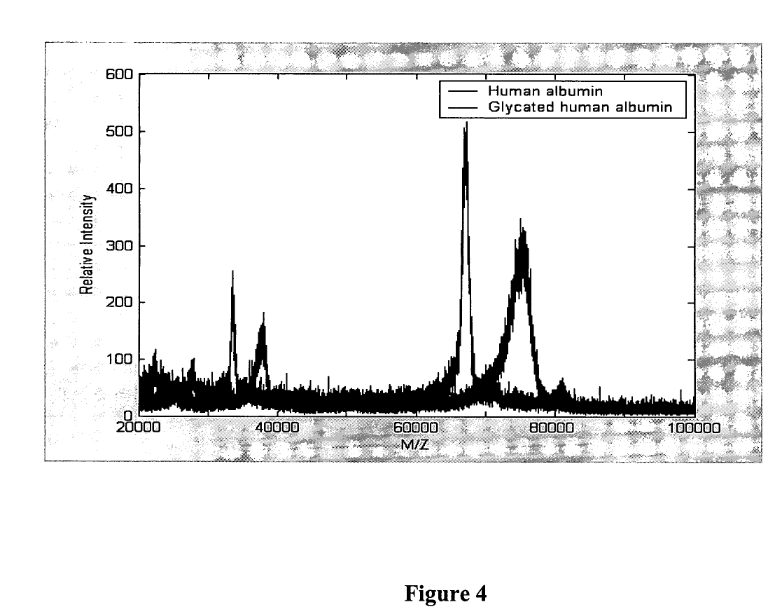

In vitro glycation of human albumin was used to produce glycated protein/peptides and to evaluate the enrichment strategy. The characterization of the reaction products by MALDI-TOF (FIG. 4) demonstrated that albumin could indeed be readily glycated. The glycation of albumin was further characterized with LC/MS/MS after tryptic digestion. Glycation on threonine was also found besides commonly reported glycation on lysine.

### Example 4

### Evaluation of Enrichment Process

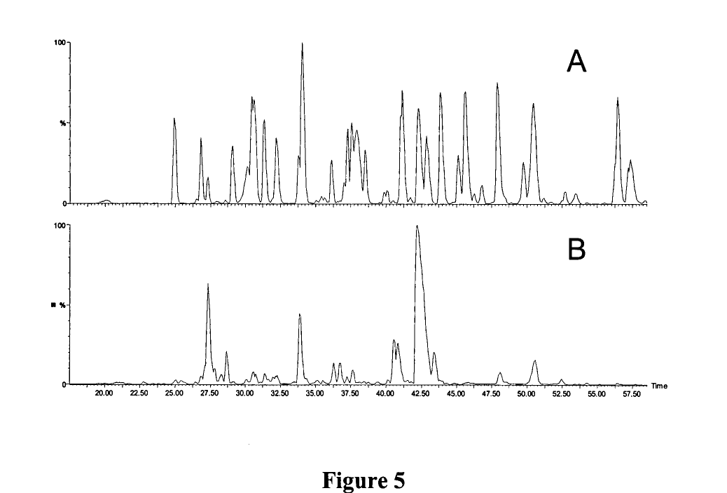

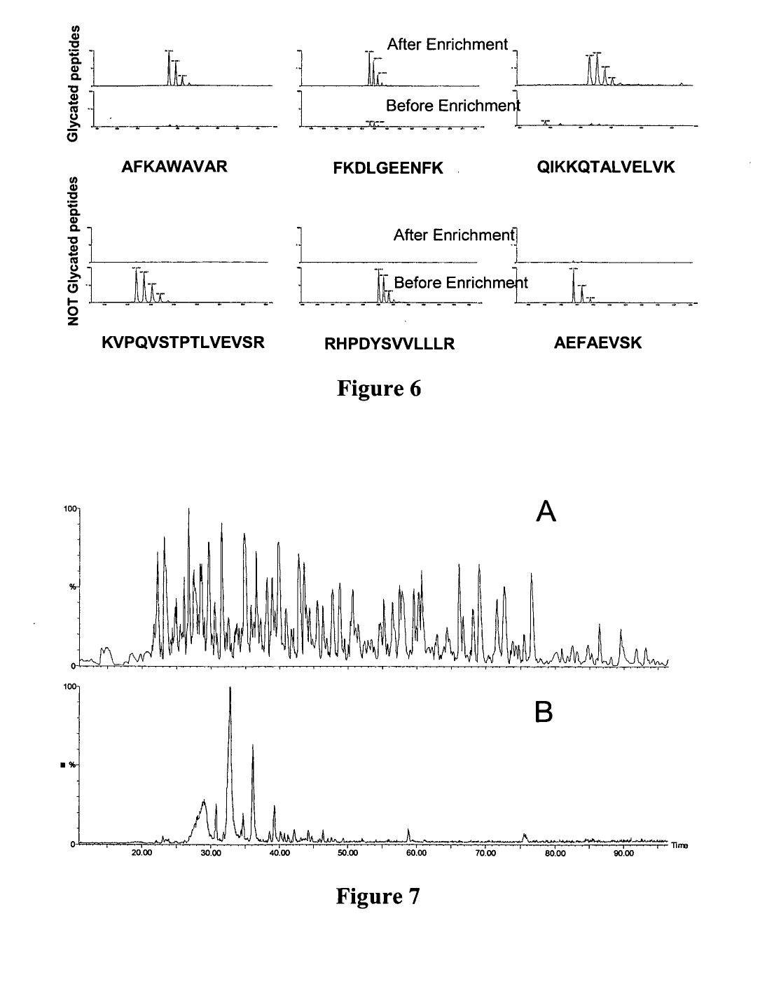

To test the selectivity and sensitivity of m-aminophenylboronic-acid-based enrichment of glycated peptides, the glycated albumin was spiked into a normal, non-glycated albumin sample at the ratio of 1 to 100 and the mixture was reduced, alkylated and digested. Immobilized m-aminophenylboronic acid on polyacrylamide beads was used to capture glycated albumin peptides. After extensive wash, the glycated peptides were released by lowering pH and analyzed by LC-MS (FIG. 5). Quantitative analysis of the LC-MS profiles shows that each of the first two cycles of enrichment amplifies the relative abundance of glycated peptides at least 100 fold and the retention of non-glycated peptides on boronic acid beads is negligible (FIGS. 6 and 7).

### Example 5

### Screening of Glycated Proteins in a Diabetic Serum Sample

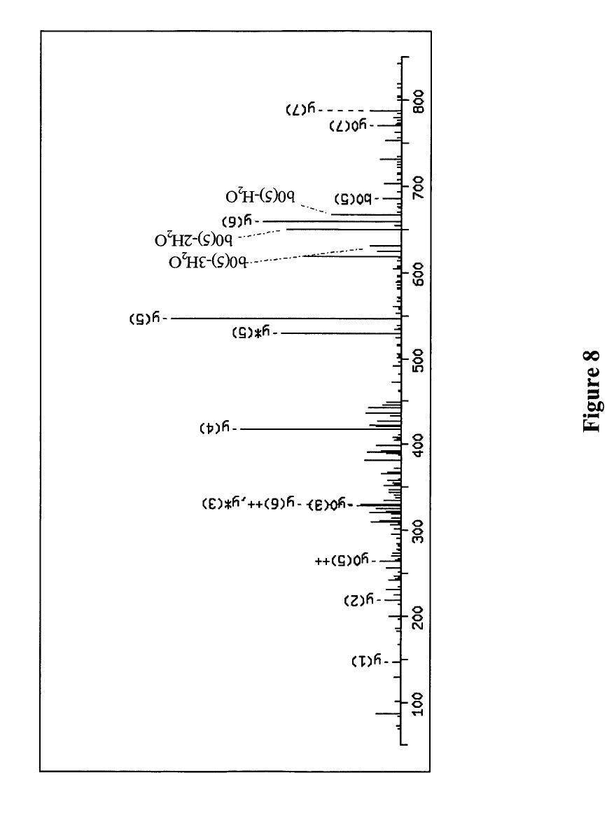

Before the enrichment methods described in Examples 1 and 2 were applied to diabetic human serum samples, N and O-linked glycans were removed to reduce possible interference during enrichment. This was done by applying a cocktail of five glycanases until the level of residual glycosylated peptides was below detectable limits. A MS/MS spectrum of a glycated peptide recovered in the enrichment procedure is shown in FIG. 8. Some identified glycation sites on serum proteins from diabetic samples are also listed in Table 2.

TABLE 2
Identified glycation sites in human diabetic serum.

| accession # | protein name | gi # |
|---|---|---|
| NP_000030.1 | apolipoprotein A-I precursor | 4557321 |
| | [Homo sapiens] | |
| NP_005134.1 | haptoglobin [Homo sapiens] | 4826762 |
| NP_001634.1 | apolipoprotein A-II precursor | 4502149 |
| | [Homo sapiens] | |
| NP_001054.1 | transferrin [Homo sapiens] | 4557871 |
| NP_000604.1 | hemopexin [Homo sapiens] | 11321561 |
| P01011 | Alpha-1-antichymotrypsin precursor | 112874 |
| | (ACT) | |
| NP_000005.1 | alpha 2 macroglobulin precursor | 4557225 |
| | [Homo sapiens] | |
| P01860 | Ig gamma-3 chain C region (Heavy | 121045 |
| | chain disease protein) (HDC) | |
| P01876 | Ig alpha-1 chain C region | 113584 |

The foregoing description of the present invention has been presented for purposes of illustration and description. Furthermore, the description is not intended to limit the invention to the form disclosed herein. Consequently, variations and modifications commensurate with the above teachings, and the skill or knowledge of the relevant art, are within the scope of the present invention. The embodiment described hereinabove is further intended to explain the best mode known for practicing the invention and to enable others skilled in the art to utilize the invention in such, or other, embodiments and with various modifications required by the particular applications or uses of the present invention. It is intended that the appended claims be construed to include alternative embodiments to the extent permitted by the prior art.

1. A method of enriching advanced glycation endproducts (AGEs) in a biological sample comprising:
a) contacting a biological sample of a mammal with a compound comprising boron wherein AGEs in the biological sample form a complex with the boron; and,
b) separating the complex from the biological sample to produce an isolate having a higher concentration of AGEs than was present in the biological sample.

2. The method of claim 1, wherein the biological sample is selected from the group consisting of cerebrospinal fluid, serum, plasma, blood, urine, feces, sweat, mucus, prostatic fluid, saliva liver tissue, pancreatic tissue, spleen tissue and combinations thereof.

3. The method of claim 1, wherein the mammal is a human.

4. The method of claim 1, wherein the compound is m-phenylboronic acid.

5. The method of claim 1, wherein the complex comprises a covalent bond between the boron and at least one oxygen present in the AGE.

6. The method of claim 1, wherein the compound comprising boron is attached to a solid support.

7. The method of claim 6, wherein the separating comprises washing the solid support to remove molecules that have not formed a complex with the compound comprising boron.

8. The method of claim 1, wherein the concentration of AGEs in the isolate is at least 50-fold greater than the concentration of AGEs in the biological sample.

9. The method of claim 1, wherein the AGE comprises a mammalian protein.

10. The method of claim 1, wherein the biological sample has been subjected to a treatment selected from the group consisting of reduction, alkylation, digestion, deglycosylation, desalting, lyophilization and combinations thereof.

11. The method of claim 1, wherein the contacting step and the separating step are conducted twice with respect to a biological sample.

12. The method of claim 1, further comprising washing the complex with a wash buffer comprising at least one chemical selected from an organic solvent, an aqueous solvent and an acidic solution.

13. The method of claim 1, further comprising eluting AGEs from the complex with the compound comprising boron.

14. The method of claim 13, wherein the eluting comprises contacting the complex with a solution having a pH of less than about pH 6 to produce an eluate comprising at least one AGE.

15. The method of claim 13, further comprising analyzing a AGE in the eluate.

16. The method of claim 15, wherein the analyzing comprises subjecting at least a portion of the eluate to an analysis methodology selected from the group consisting of protein sequencing, immunoassay, hybridization, enzyme assay, liquid chromatography (LC), mass spectroscopy (MS), gas chromatography (GC), electrospray ionization—time of flight (ESI-TOF) spectroscopy, matrix assisted laser desorption/ionization—time of flight (MALDI-TOF) spectroscopy, chromatographic separation, 2-D gel separation, immunoassay, competitive inhibition assays and combinations thereof, to produce a data set that can be compared to a reference data set.

17. The method of claim 16, wherein the reference data set is produced by analyzing a biological sample from at least one mammal that has not been diagnosed with diabetes.

18. A method of producing an antibody useful in the clinical evaluation or progression of diabetes or metabolic syndrome comprising:
a) contacting a biological sample of a mammal known to have diabetes or metabolic syndrome with a compound comprising boron wherein AGEs in the biological sample form a complex with the boron;
b) separating the complex from the biological sample to produce an isolate having a higher concentration of AGEs than was present in the biological sample;
c) immunizing a host animal with AGEs; and,
d) isolating antibodies to the AGEs from the host animal.

19. A method of diagnosing diabetes or metabolic syndrome in a mammal comprising analyzing a biological sample from the mammal for a biomarker of diabetes or metabolic syndrome identified by the method comprising:
a) contacting a biological sample of a mammal known to have diabetes or metabolic syndrome with a compound comprising boron wherein AGEs in the biological sample form a complex with the boron;
b) separating the complex from the biological sample to produce an isolate having a higher concentration of AGEs than was present in the biological sample; and,
c) analyzing the AGEs for the presence of biological markers that correlate with the incidence of diabetes or metabolic syndrome.

20. A method of identifying a therapeutic target for the treatment or prevention of diabetes comprising:
a) contacting a biological sample of a mammal known to have diabetes or metabolic syndrome with a compound comprising boron wherein AGEs in the biological sample form a complex with the boron;
b) separating the complex from the biological sample to produce an isolate having a higher concentration of AGEs than was present in the biological sample; and,
c) analyzing the AGEs for the presence of biological markers that correlate with the incidence of diabetes or metabolic syndrome.

21. A method of monitoring the progression of diabetes or metabolic syndrome in a mammal comprising analyzing a biological sample from the mammal for a biomarker of diabetes or metabolic syndrome identified by the method comprising:
a) contacting a biological sample of a mammal known to have diabetes or metabolic syndrome with a compound comprising boron wherein AGEs in the biological sample form a complex with the boron;
b) separating the complex from the biological sample to produce an isolate having a higher concentration of AGEs than was present in the biological sample; and,
c) analyzing the AGEs for the presence of biological markers that correlate with the incidence of diabetes or metabolic syndrome.
